(19) United States
(12) Patent Application Publication
(10) Pub. No.: US 2016/0194258 A1
(43) Pub. Date: Jul. 7, 2016

(54) METHOD FOR PRODUCING HYDROCARBON PRODUCTS

(71) Applicant: LINDE AKTIENGESELLSCHAFT, München (DE)

(72) Inventors: Stefanie Walter, Seehausen (DE); Helmut Fritz, Munchen (DE)

(21) Appl. No.: 14/916,157

(22) PCT Filed: Sep. 4, 2014
(86) PCT No.: PCT/EP2014/068842

§ 371 Date: Mar. 2, 2016

(30) Foreign Application Priority Data
Sep. 5, 2013 (DE) 10 2013 014 802.2
Sep. 5, 2013 (DE) 10 2013 014 867.7
Sep. 25, 2013 (EP) 13004660.0
Sep. 25, 2013 (EP) 13004661.8

(51) Int. Cl. C07C 4/04; C07C 41/09; C07C 5/03; C07C 41/34; C07C 5/27; C07C 5/25

(52) U.S. Cl. CPC C07C 4/04; C07C 5/2767; C07C 5/25; C07C 5/03; C07C 41/34; C07C 41/09

(57) ABSTRACT

A method for producing hydrocarbon products is proposed which comprises providing a C4 hydrocarbon stream (C4) that predominantly comprises branched and unbranched hydrocarbons each having four carbon atoms, and an n-C4 partial stream (n-C4), which predominantly comprises unbranched hydrocarbons with four carbon atoms and an iso-C4 partial stream (i-C4) which predominantly comprises branched hydrocarbons with four carbon atoms, from the C4 hydrocarbon stream (C4) or a stream derived therefrom. It is envisaged that at least part of the n-C4 partial stream (n-C4) or a stream derived therefrom should be cracked at a cracking severity at which not more than 92% of n-butane contained in the n-C4 partial stream (n-C4) or in the derived stream is converted.

The invention relates to a method for producing hydrocarbon products according to the precharacterising clauses of the independent claims.

### PRIOR ART

Methods and apparatus for steam cracking hydrocarbons are known and are described for example in the article “Ethylene” in Ullmann's Encyclopedia of Industrial Chemistry, online since 15 Apr. 2007, DOI 10.1002/14356007.a10_045.pub2.

U.S. Pat. No. 3,922,216 discloses a method in which a hydrocarbon stream which predominantly contains hydrocarbons with two to five carbon atoms is combined, after the removal of isobutene, with a hydrocarbon stream containing hydrocarbons with nine or more carbon atoms. The stream thus formed is subjected to a steam cracking process. EP 2 062 865 A1 discloses a method for producing ethylene, propylene and isoprene from light hydrocarbons in which iso-butane is separated from a butane fraction which may optionally contain ethane. The remainder can be reacted in one or more cracking zones in a steam cracking process. Processes of the same generic type are described in DE 28 05 179 A1, U.S. Pat. No. 6,743,958 B2, U.S. Pat. No. 5,523,502, US 2011/112345 A1 and U.S. Pat. No. 4,091,046.

In more recent steam cracking methods and apparatus, mild cracking conditions are increasingly used (see below), because these produce in particular so-called high value products, for example propylene and butadiene, in improved yields, as explained hereinafter. However, at the same time, the conversion of the furnace feed is decreased under mild cracking conditions, with the result that compounds contained therein are 3o found in the cracking gas in comparatively large amounts and lead to “dilution” of the high value products.

The problem of the invention is to remedy this and to retain the advantages of the mild cracking conditions while avoiding the disadvantages. In particular, by reducing the diluting effect mentioned above, the concentration and quantity of the high value products, particularly 1,3-butadiene, should be increased.

### DISCLOSURE OF THE INVENTION

This problem is solved by a method for producing hydrocarbon products having the features of the independent claims. Preferred embodiments are the subject of the dependent claims and of the description that follows.

Before the features and advantages of the present invention are described, their basis and the terminology used will be explained.

Steam cracking processes are carried out on a commercial scale almost exclusively in tubular reactors in which individual reaction tubes (in the form of coiled tubes, so-called coils) or groups of corresponding reaction tubes can be operated even under different cracking conditions. Reaction tubes or sets of reaction tubes and possibly also tube reactors operated under uniform cracking conditions are hereinafter referred to as “cracking furnaces” in each case. A cracking furnace, in the terminology as used herein, is thus a constructive unit used for steam cracking which exposes a furnace feed to identical or comparable cracking conditions. A steam cracking apparatus may comprise one or more cracking furnaces of this kind.

The term “furnace feed” used here denotes one or more liquid and/or gaseous streams which are fed into one or more cracking furnaces. Also, streams obtained by a corresponding steam cracking process as explained hereinafter may be recycled into one or more cracking furnaces and used again as a furnace feed. A large number of hydrocarbons and hydrocarbon mixtures from ethane to gas oil up to a boiling point of typically 600° C. are suitable as furnace feeds.

A furnace feed may consist of a so-called “fresh feed”, i.e. of a feed which is prepared outside the apparatus and is obtained for example from one or more petroleum fractions, petroleum gas components with two to four carbon atoms and/or petroleum gas condensates. A furnace feed may also consist of one or more so-called “recycle streams”, i.e. streams that are produced in the apparatus itself and are recycled into a corresponding cracking furnace. A furnace feed may also consist of a mixture of one or more fresh feeds with one or more recycle streams.

The furnace feed is at least partly converted in the respective cracking furnace and leaves the cracking furnace as a so-called “raw gas”, which, as explained hereinafter with reference to FIGS. 1A and 1B, may be subjected to a series of after-treatment steps. These after-treatment steps encompass, first of all, processing of the raw gas, for example by quenching, cooling and drying, so as to obtain a so-called “cracking gas”. Occasionally the raw gas is also referred to as cracking gas.

Current methods include in particular the separation of the cracking gas into a number of fractions based on the different boiling points of the components obtained. In the art, abbreviations are used for these which indicate the carbon number of the hydrocarbons that are predominantly or exclusively contained. Thus, a “C1 fraction” is a fraction which predominantly or exclusively contains methane (but according to convention also contains hydrogen in some cases, then also called “Ciminus fraction”). A “C2 fraction” on the other hand predominantly or exclusively contains ethane, ethylene and/or acetylene. A “C3 fraction” predominantly contains propane, propylene, methyl acetylene and/or propadiene. A “C4 fraction” predominantly or exclusively contains butane, butene, butadiene and/or butyne, while the respective isomers may be present in different amounts depending on the source of the C4 fraction. The same also applies to a “C5 fraction” and the higher fractions. Several such fractions may also be combined in one process and/or under one heading. For example, a “C2plus fraction” predominantly or exclusively contains hydrocarbons with two or more carbon atoms and a “C2minus fraction” predominantly or exclusively contains hydrocarbons with one or two carbon atoms.

Liquid and gaseous streams may, in the terminology of the art, be rich in or poor in one or more components, “rich” indicating a content of at least 90%, 95%, 99%, 99.5%, 99.9%, 99.99% or 99.999% and “poor” indicating a content of at most 10%, 5%, 1%, 0.1%, 0.01% or 0.001% on a molar, weight or volume basis. The term “predominantly” denotes a content of at least 50%, 60%, 70%, 80% or 90% or corresponds to the term “rich”. Liquid and gaseous streams may also, in the terminology as used herein, be enriched or depleted in one or more components, these terms relating to a corresponding content in a starting mixture from which the liquid or gaseous stream was obtained. The liquid or gaseous stream is “enriched” if it contains at least 1.1 times, 1.5 times, 2 times, 5 times, 10 times, 100 times or 1,000 times the amount, “depleted” if it contains at most 0.9 times, 0.5 times, 0.1 times, 0.01 times or 0.001 times the amount of a corresponding component, based on the starting mixture.

A stream may be “derived” from another stream, for example by dilution, concentration, enrichment, depletion, separation or reaction of any desired components, by separating steps or also by combining with at least one other stream. A derived stream may also be formed by dividing an initial stream into at least two partial streams, each partial stream or a residual stream remaining after the separation of another stream being a derived stream of this kind.

The above-mentioned “cracking conditions” in a cracking furnace encompass inter alia the partial pressure of the furnace feed, which may be influenced by the addition of different amounts of steam and the pressure selected in the cracking furnace, the dwell time in the cracking furnace and the temperatures and temperature profiles used therein. The furnace geometry and configuration also play a part. To produce ethylene a cracking furnace is operated for example at a furnace entry temperature of 500 to 680° C. and at a furnace exit temperature of 775 to 875° C. The “furnace entry temperature” is the temperature of a gas stream at the start of a reaction tube and the “furnace exit temperature” is the temperature of a gas stream at the end of a reaction tube. Typically, the latter is the maximum temperature to which the gas stream in question is heated. It is mixed with the furnace feed at a pressure of 165 to 225 kPa, measured at the furnace exit, in a ratio of typically 0.25 to 0.85 kg/kg. The values specifically used are dependent on the particular furnace feed used and the desired cracking products.

As the values mentioned influence one another at least partially, the term “cracking severity” has been adopted to characterise the cracking conditions. For liquid furnace feeds the cracking severity can be described by means of the ratio of propylene to ethylene (P/E) or as the ratio of methane to propylene (M/P) in the cracking gas based on weight (kg/kg). The P/E and M/P ratios are directly dependent on the temperature, but, unlike the real temperature in or at the exit from a cracking furnace, they can be measured much more accurately and be used for example as a control variable in a corresponding regulating process. The P/E ratio is however only of limited use in characterising the cracking severity in gaseous furnace feeds or in compounds with two to four carbon atoms.

For gaseous furnace feeds the reaction or conversion of a particular component of the furnace feed may be specified as a measure of the cracking severity. The term reaction or conversion is used in the manner conventional in the art (cf. for example the above-mentioned article “Ethylene” in Ullmann's Encyclopedia of Industrial Chemistry). In particular, for the C4 fractions or C4 partial streams used in the present case, it is useful to describe the cracking severity in terms of the conversion of key components such as n-butane and iso-butane.

The cracking severities or cracking conditions are “severe” if n-butane in a corresponding fraction is converted by more than 92%. Under even more severe cracking conditions, n-butane is optionally converted by more than 93%, 94% or 95%. Typically, there is no 100% conversion of n-butane. The upper limit of the “severe” cracking severities or cracking conditions is therefore 99%, 98%, 97% or 96% conversion of n-butane, for example. The cracking severities or cracking conditions are “mild”, on the other hand, if n-butane is converted by less than 92%. At less than 91%, less than 90%, less than 89%, less than 88% or less than 87% conversion of n-butane, increasingly milder cracking severities or cracking conditions are present. At less than 86% conversion of n-butane the cracking severities or cracking conditions are referred to as “very mild”. Very mild cracking severities or cracking conditions also encompass, for example, a conversion of n-butane of less than 85%, 84%, 83%, 82%, 81%, 80%, 79%, 78%, 77%, 76%, 75%, 70% or 65% and more than 50% or 60%.

The cracking severities or cracking conditions are also “severe” when iso-butane in a corresponding fraction is converted by more than 91%. Under even more severe cracking conditions iso-butane is optionally converted by more than 92%, 93% or 94%. Typically, there is no 100% conversion of iso-butane either. The upper limit of the “severe” cracking severities or cracking conditions is therefore at 99%, 98%, 97% or 96% conversion of iso-butane, for example. The cracking severities or the cracking conditions are, however, “mild” if iso-butane is converted by less than 91%. At less than 90%, less than 89%, less than 88%, less than 87% or less than 86% conversion of iso-butane, milder cracking severities or cracking conditions are increasingly obtained. At less than 83% conversion of iso-butane the cracking severities or cracking conditions are designated here as “very mild”. Very mild cracking severities or cracking conditions also include for example a conversion of iso-butane of less than 82%, 81%, 80%, 79%, 78%, 77%, 76%, 75% or 70% and more than 45% or 50%. The above-mentioned cracking severities or cracking conditions are correlated in particular with the furnace exit temperature at the end of the conversion tube or cracking furnaces used, as described above. The higher this temperature, the more “severe”, and the lower the temperature, the “milder” the cracking severities or cracking conditions.

It should also be understood that the conversion of other components does not have to be identical to that of n- and iso-butane. If, for example, 1- and 2-butene are cracked together with n-butane, these are typically converted to a greater extent than n-butane. Conversely, iso-butene is converted to a lesser extent than iso-butane, if it is converted together with the latter. A percentage conversion of a key component, in this case n-butane or iso-butane, is therefore associated with a furnace exit temperature and the respective percentage conversions of the other components in the feedstock. This furnace exit temperature is in turn dependent on the cracking furnace, among other things. The difference between the respective percentage conversions is dependent on a number of other factors.

### ADVANTAGES OF THE INVENTION

The present invention starts from a method for producing hydrocarbon products in which a hydrocarbon stream is provided which comprises predominantly, i.e. at least 80%, branched and unbranched hydrocarbons with four carbon atoms (referred to hereinafter and in accordance with the usual terminology as C4 fraction or C4 hydrocarbon stream, abbreviated to C4, which is also used as the reference numeral in the Figures). To this extent the method according to the invention corresponds for example to known methods of producing hydrocarbon products by steam cracking, in which a C4 hydrocarbon stream of this kind is separated from a cracking gas which has optionally been further treated. This may take place in known apparatus in a so-called debutanizer (although this also separates all the other hydrocarbons with four carbon atoms from a corresponding hydrocarbon stream). Details of this are shown in the above-mentioned article “Ethylene” in Ullmann's Encyclopedia of Industrial Chemistry and illustrated with reference to FIGS. 1A and 1B.

However, the invention is not restricted to the use of C4 hydrocarbon streams provided by steam cracking and subsequent process steps, but is equally suitable for C4 hydrocarbon streams produced at least partly using other methods, for example by refinery processes. For example, the invention may be used with C4 streams which have not been steam-cracked beforehand and are only subsequently fed into a corresponding steam cracking process. These may be, for example, petroleum fractions, petroleum gas components with two to four carbon atoms, petroleum gas condensates and the like or products obtained from refinery conversion processes. In particular, a C4 hydrocarbon stream of this kind may be subjected to one or more processes by means of which compounds contained in the C4 hydrocarbon stream are reacted and/or separated.

The invention now envisages recovering a partial stream with predominantly, i.e. at least 80%, unbranched hydrocarbons with four carbon atoms (that is, n-C4 compounds, therefore referred to as n-C4 fraction or n-C4 partial stream; abbreviated to n-C4 which is also the reference numeral used in the Figures) and a partial stream with predominantly branched hydrocarbons with four carbon atoms (that is, iso-C4 compounds, therefore referred to as iso-C4 fraction or iso-C4 partial stream; abbreviated to i-C4 which is also the reference numeral used in the Figures) from this C4 hydrocarbon stream or a stream derived therefrom.

The invention envisages cracking at least part of the n-C4 partial stream or a stream derived therefrom at a cracking severity at which n-butane contained in the n-C4 partial stream is converted to at least 50% and less than 92%. Thus, mild or very mild cracking severities or cracking conditions are used. These may also correspond for example to less than 90%, 88%, 86%, 84%, 82%, 80%, 78%, 76%, 74%, 72%, 70% or 65%, but more than 50% or 60% conversion of n-butane, for example. Under correspondingly mild cracking conditions, more desirable products compared with the fresh feed are obtained, such as butadiene and propylene, so that the yield is increased and the product spectrum is improved.

The “cracking” comprises feeding the n-C4 partial stream or a stream derived therefrom (or a corresponding part thereof) on its own or together with other streams, optionally after previous combining to form a combined stream, into a cracking furnace according to the definition provided hereinbefore and removing a cracking gas from the cracking furnace.

Advantageously, iso-C4 compounds contained in the iso-C4 partial stream are also at least partially reacted by a skeleton isomerisation process to form n-C4 compounds, for example before a corresponding partial stream is fed into a cracking furnace (see below) and/or before its removal as product and/or before further separation of the iso-C4 partial stream. A corresponding skeleton isomerisation process comprises using suitable catalysts and reaction conditions, as disclosed for example in U.S. Pat. No. 6,743,958 B2 or U.S. Pat. No. 6,916,448 B2:

Skeleton isomerisation may be carried out using aluminium oxide catalysts (in which γ-aluminium oxide may be used as adsorbent, as catalyst support and/or as the catalyst itself). Activated and/or steam-treated aluminium oxide may also be used, for example, as described in U.S. Pat. No. 3,558,733. Moreover, compounds containing titanium or boron may be used, particularly in conjunction with n- or y-aluminium oxide, as described in U.S. Pat. No. 5,321,195 and U.S. Pat. No. 5,659,104. Other compounds that may be used are halogenated aluminium oxides, as disclosed for example in U.S. Pat. No. 2,417,647, bauxites or zeolites. The use of molecular sieves of microporous structure is also known in this context, for example from EP 0 523 838 A1, EP 0 501 577 A1 and EP 0 740 957 A1.

These may also form active phases of catalysts. Aluminium oxide-based catalysts are generally used in the presence of water at temperatures of 200 to 700° C. and pressures of 0.1 to 2 MPa, particularly at temperatures of 300 to 570° C. and pressures of 0.1 to 1 MPa. Other reaction conditions for the skeleton isomerisation may be inferred from the publications mentioned above.

n-C4 compounds obtained by skeleton isomerisation are advantageously also subjected to the mild cracking conditions mentioned hereinbefore; in particular they may be combined with the n-C4 partial stream which is obtained according to the invention from the C4 hydrocarbon stream. During the skeleton isomerisation (initially) unconverted iso-C4 compounds may be at least partially removed from a corresponding apparatus, subjected to severe cracking conditions and/or subjected once again to any of the processes described hereinbefore or hereinafter. In particular it may be advantageous to subject at least some of these iso-C4 compounds once more to the skeleton isomerisation described until total conversion has been achieved. It may also be advantageous to combine the whole of the stream obtained in the skeleton isomerisation which contains n-C4 compounds and (unconverted) iso-C4 compounds with the C4 hydrocarbon stream used as the starting stream. In this way the n-C4 compounds obtained can be separated using the same apparatus that is already provided for recovering the n-C4 partial stream and the iso-C4 partial stream. The introduction may take place at any desired point, i.e. upstream or downstream of any desired processes carried out before the recovery of the n-C4 partial stream and the iso-C4 partial stream, so that these compounds obtained in the skeleton isomerisation can also be used.

The steam cracking of C4 fractions of different origins is known in the art. The cracking results can be reliably predicted with the tools available. As a rule, they are present as mixtures of branched and unbranched C4-compounds. In the fresh feeds mentioned previously, these predominantly comprise paraffinic compounds, while in recycling streams from steam cracking processes or in products of other treatment processes (e.g. from refineries) they predominantly comprise olefinic compounds.

Particularly when cracking naphthas under mild cracking conditions or with a high proportion of C4 fresh feeds, the proportion of C4 fraction obtained from the corresponding cracking gas is also great and in particular has a relatively low concentration of 1,3-butadiene and optionally other high value products which are to be extracted from the C4 fraction. As a result, the recovery of 1,3-butadiene is uneconomical.

Particularly when steam cracking hydrocarbons under unusually mild conditions there is thus a substantial increase in the amount of some product fractions, as already mentioned, and a consequent reduction in the concentration of high value products present (dilution effect). This makes the recovery of the high value products more difficult or more expensive.

The invention is based on the finding that branched C4 compounds in the cracking furnace contribute to the formation of 1,3-butadiene to a minor extent by reason of their structure. A relatively high methane formation from such compounds is unavoidable, particularly when the branched C4 compounds are recycled until fully converted. Thus, if C4 hydrocarbon streams with branched and unbranched C4 compounds as a whole are cracked under mild or even very mild conditions, this results in C4 fractions of relatively large streams with, at the same time, a low concentration of 1,3-butadiene.

This effect is countered according to the invention by obtaining the n-C4 partial stream from the C4 hydrocarbon stream before the steam cracking, for example by distillation, and cracking it, or the n-C4 compounds predominantly contained therein, but preferably not the iso-C4 compounds, under the mild conditions specified. The iso-C4 compounds which, as already explained, are separated off in an iso-C4 partial stream and are still present as such, even after subsequent processes such as skeleton isomerisation, may be obtained at least partly as products.

Advantageously, however, the iso-C4 partial stream or a stream derived therefrom, for example a stream or partial stream which is present downstream of a hydrogenation process (see below) or a skeleton isomerisation process, is at least partly cracked at a high cracking severity. In such a case, very severe cracking conditions are used, in contrast to the n-C4 partial stream, which is cracked particularly mildly.

Advantageously, therefore, the iso-C4 partial stream or a stream derived therefrom is at least partially cracked at a cracking severity at which iso-butane contained therein is converted by more than 91%. The “cracking” here also includes feeding the iso-C4 partial stream or the stream derived therefrom (or a corresponding part of it), on its own or in conjunction with other streams, optionally after combining them beforehand to form a combined stream, into a cracking furnace according to the definition provided hereinbefore and removing a cracking gas from the cracking furnace.

The cracking severities used for cracking the iso-C4 partial stream are thus higher than the cracking severities used for cracking the n-C4 partial stream, the terms “higher” and “lower” relating to one another. Thus, advantageously, at least part of the iso-C4 partial stream or a stream derived therefrom is cracked at a first cracking severity and at least a part of the n-C4 partial stream or a stream derived therefrom is cracked at a second cracking severity, the first cracking severity being higher than the second, or the second being lower than the first.

Thanks to the invention, the disadvantages of mild cracking are reduced or eliminated completely, while retaining its advantages, i.e. the amount of C4 fraction is reduced and as a result the concentration of the target product, in this case particularly 1,3-butadiene, is increased. The specific extraction effort is reduced.

The core of the invention thus consists in minimising the C4 fraction as a whole by the selective use of mild, particularly very mild cracking conditions on the n-C4 compounds, so as to obtain the high selectivities, e.g. in respect of 1,3-butadiene, which can be achieved thereby. Advantageously, this results in a controlled increase in the structural conversion of iso-C4 compounds, for example by severe cracking after previous separation of the n-C4 compounds.

The invention further envisages, in order to improve the separation of the iso- and n-C4 compounds, at least partially reacting any i-butene present to form 2-butene in the C4 hydrocarbon stream, i.e. the hydrocarbon stream which predominantly comprises branched and unbranched hydrocarbons with four carbon atoms. A hydroisomerisation process is used for this purpose.

The C4 hydrocarbon stream or at least a stream derived from the C4 hydrocarbon stream (i.e. separated off, combined with another stream, etc.) is fed into at least one hydroisomerisation reactor. A C4 hydrocarbon stream rich in 2-butene and at the same time depleted in i-butene is obtained in which other components may also have been reacted by the hydroisomerisation process. For example, any remaining traces of butadiene may be eliminated in this way. However, the components that have not been reacted by the hydroisomerisation process are still present in this stream. A stream of this kind is also referred to as the “offstream” of the hydroisomerisation process (or of a hydroisomerisation reactor used in it). It is a stream derived from the C4 hydrocarbon stream.

It may also be advantageous to feed at least one other stream, particularly a stream containing butyne (C4-acetylene) and/or hydrocarbons each having five carbon atoms, into the C4 hydrocarbon stream, which predominantly contains branched and unbranched hydrocarbons each having four carbon atoms, before or after the hydroisomerisation or another process. For example, C5 compounds coextracted during a butadiene extraction (see below) may be used, thus putting them to good use.

This hydroisomerisation ensures a conversion of the 1-butene to form 2-butene in a corresponding C4 hydrocarbon stream, thus making it significantly easier to separate the iso-C4 from the n-C4 compounds. This is a result of the significantly higher boiling point of the 2-butene or its two isomers (cis-2-butene: 3.72° C. at atmospheric pressure;

trans-2-butene: 0.88° C. at atmospheric pressure) compared with 1-butene (−6.26° C. at atmospheric pressure). 1-Butene, by contrast, because of its boiling point, cannot in practice be distillatively separated from iso-butene with its virtually identical boiling point (−6.9° C. at atmospheric pressure). The basic concept of the present invention, namely separating off the iso-C4 compounds which are disadvantageous to the very mild cracking conditions before the mild cracking, can therefore be implemented more easily and cheaply using the hydroisomerisation. In addition, C4-acetylenes can be reacted to form n-butenes by the hydroisomerisation.

Hydroisomerisation processes are known per se and are described for example in EP 1 871 730 B1, US 2002/169346 A1, U.S. Pat. No. 6,420,619 B1, U.S. Pat. No. 6,075,173 and WO 93/21137 A1. In such processes, a corresponding stream is typically passed through a hydroisomerisation reactor in the presence of a hydroisomerisation catalyst. The hydroisomerisation reactor is typically embodied as a solid bed reactor. Preferably, the hydroisomerisation process results in the maximum possible conversion of 1-butene to 2-butene. However, the conversion that is actually carried out depends on economic considerations, among other things.

It is particularly advantageous if the iso-C4 partial stream, i.e. the one comprising predominantly branched hydrocarbons with four carbon atoms, or a stream derived therefrom, is at least partially subjected to a hydrogenation process, optionally before a subsequent steam cracking. During this process the iso-butene present (olefinic) is reacted at least partially to form iso-butane (paraffinic). In the subsequent cracking process, i.e. at the higher cracking severity, the iso-butane can be reacted more easily, or to form more easily utilisable products. This makes it possible to reduce the quantity of the C4 fraction still further and thereby concentrate the target products, as mentioned hereinbefore.

For hydrogenating and hydroisomerising olefins or olefin-containing hydrocarbon mixtures, numerous catalytic methods are known from the prior art, which can also be used within the scope of the present invention. Hydration catalysts have, as a hydrogenation-active component, one or more elements of the 6^{th}, 7^{th} or 8^{th} sub-group of the Periodic Table in elemental or bound form. Typically, noble metals of the 8^{th} sub-group in elemental form are used as hydroisomerisation catalysts. They may be doped with different additives in order to influence specific catalyst properties, for example service life, resistance to specific catalyst poisons, selectivity or regenerability. The hydrogenation and hydroisomerisation catalysts often contain the active component on supports, for example mordenites, zeolites, Al₂O₃ modifications, SiO₂ modifications and so on.

Generally, reaction temperatures of 150 to 250° C. are used for the extensive hydrogenation of the olefins. The hydroisomerisation is carried out at remarkably lower temperatures. The thermodynamic equilibrium is towards the internal olefins, in this case 2-butene, at these lower temperatures.

The process variants described above may encompass at least partially forming the above-mentioned C4 hydrocarbon stream from at least one cracking gas stream which is produced during the steam cracking according to the invention of the n-C4 partial stream or corresponding proportions thereof and/or streams derived therefrom, optionally together with fresh feed.

However, the C4 hydrocarbon stream may also be formed at least partly from a cracking gas which is obtained by steam cracking a fresh feed, or from an uncracked fresh feed. These alternatives make it possible to achieve a very flexible adjustment of the desired content of the C4 hydrocarbon stream in terms of the individual C4 compounds.

The steam cracking is advantageously carried out within the scope of the present invention using a quantity of steam of 0.4 kg/kg, particularly 0.2 to 0.7 kg/kg, for example 0.3 to 0.5 kg/kg , with identical or different values in the cracking furnaces used. Corresponding values may in particular also be adapted to other cracked feeds.

If different cracking severities are used these may advantageously be adjusted in at least one cracking furnace in each case, which is supplied with at least one other furnace feed. For example, a cracking furnace designed for a corresponding throughput may be used which is operated at a lower cracking severity and in which, besides the n-C4 stream, a “regular” fresh feed is also mildly cracked. In a corresponding variant of the process the iso-C4 stream may also be cracked on its own in a cracking furnace operated at the higher cracking severity. In certain cases, however, for example when similar cracking furnaces are used for reasons of cost, it may be more sensible to crack the iso-C4 stream severely, together with a fresh feed.

It will be understood that the entire iso-C4 stream, if formed, does not have to be cracked under severe conditions. It is also possible to crack part of the iso-C4 stream under mild conditions. However, at least some will be cracked severely, thus achieving a corresponding reduction in quantity, as explained hereinbefore.

As mentioned above, the particular purpose of the present invention is to improve a method in which 1,3-butadiene is separated from the hydrocarbon stream. All the known methods for extracting 1,3-butadiene are suitable for this purpose.

Other advantages may be obtained if, after separation of the 1,3-butadiene, isobutene contained in the hydrocarbon stream is at least partly reacted to form a tert-butylether and this is also extracted, for example before hydroisomerisation. The preparation of methyl-tert-butylether (2-methoxy-2-methylpropane, MTBE) is known in principle. MTBE is produced industrially with acid catalysis from isobutene and methanol, which is added to the hydrocarbon stream. MTBE is mainly used as an anti-knocking agent, but is also increasingly used as a solvent and extraction agent in organic chemistry. Ethanol yields ethyl-tert-butylether. Other alcohols may also be used.

The apparatus according to the invention is advantageously designed to perform a process as described hereinbefore.

The at least one separating device advantageously comprises at least one separating column and the steam cracking device advantageously comprises at least two cracking furnaces which are designed to operate at different cracking severities.

The invention is hereinafter explained relative to the prior art with reference to the appended Figures.

### BRIEF DESCRIPTION OF THE DRAWINGS

FIG. 1A schematically shows the course of a process for producing hydrocarbons according to the prior art.

FIG. 1B schematically shows the course of a process for producing hydrocarbons according to the prior art.

FIG. 2 schematically shows the course of a process for producing hydrocarbons according to one embodiment of the invention.

FIG. 3 schematically shows the course of a process for producing hydrocarbons according to one embodiment of the invention.

FIG. 4 schematically shows the course of a process for producing hydrocarbons according to one embodiment of the invention.

FIG. 5 schematically shows the course of a process for producing hydrocarbons according to one embodiment of the invention.

FIG. 6 schematically shows the course of a process for producing hydrocarbons according to one embodiment of the invention.

In the Figures, corresponding elements have been given identical reference numerals and are not explained repeatedly, in the interests of clarity.

### DETAILED DESCRIPTION OF THE DRAWINGS

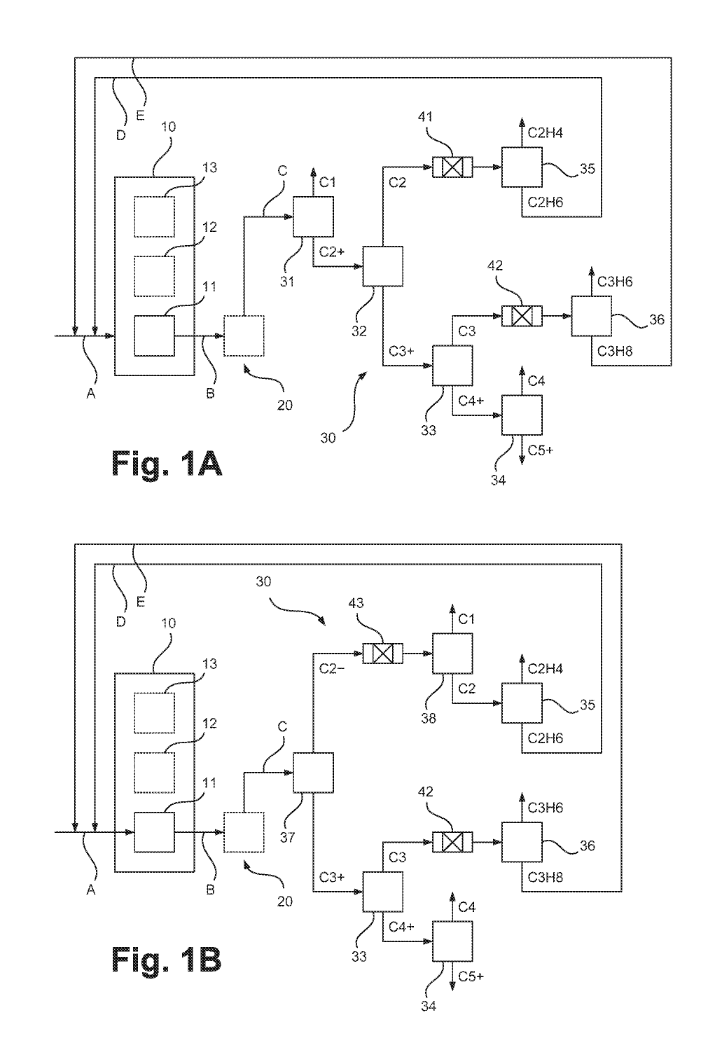

FIG. 1A shows the course of a method of producing hydrocarbons according to the 3o prior art in the form of a schematic flow diagram. The core of the method here is a steam cracking process 10 which can be carried out using one or more cracking furnaces 11 to 13. Only the operation of the cracking furnace 11 is described hereinafter, the other cracking furnaces 12 and 13 may operate in a corresponding manner.

The cracking furnace 11 is charged with a stream A as the furnace feed, and this may be at least partially a so-called fresh feed which is provided from sources outside the apparatus, and at past partially a so-called recycle stream which is obtained in the method itself, as explained below. The other cracking furnaces 12 and 13 may also be charged with corresponding streams. Different streams may also be fed into different cracking furnaces 11 to 13, one stream may be divided between several cracking furnaces or several partial streams may be combined to form one combined stream which is fed for example as stream A into one of the cracking furnaces 11 to 13.

As a result of steam cracking in the steam cracking process 10 a raw gas stream B is obtained which occasionally already at this point referred to herein as a cracking gas current. The crude gas stream B is prepared in a series of preparation stages (not shown) of a preparation process 20, subjected to a so-called oil quench, for example, pre-fractionated, compressed, cooled further and dried.

The correspondingly treated stream B, the actual cracking gas C, is then subjected to a separation process 30. In this process a number of fractions are obtained which, as explained hereinbefore, are named according to the carbon number of the hydrocarbons that they predominantly contain. The separation process 30 shown in FIG. 1A operates according to the principle of “Demethanizer First”, a separation process according to the principle of “Deethanizer First” is shown in FIG. 1B.

In the separation process 30 a C1- or C1minus fraction (designated C1) which may also contain hydrogen, unless it has already been removed beforehand, is first separated in gas form from the cracking gas C in a first separating unit 31 (the so-called demethanizer). It is typically used as a combustion gas. A liquid C2plus fraction (reference numeral C2+) remains which is transferred into a second separating unit 32 (the so-called Deethanizer).

In the second separating unit 32 a C2 fraction (reference numeral C2) is separated off in gaseous form from the C2plus fraction and subjected for example to a hydrotreatment process 41 in order to react any acetylene present to ethylene. Then the C2 fraction is separated in a C2 separating unit 35 into ethylene (reference numeral C2H4) and ethane (reference numeral C2H6). The latter can be subjected to the steam cracking process 10 again as a recycle stream D in one or more cracking furnaces 11 to 13. In the example shown the recycle streams D and E are added to the stream A. The recycle streams D and E and the stream A can also be fed into different cracking furnaces 11 to 13.

In the second separating unit 32 a liquid C3plus fraction (reference numeral C3+) remains, which is transferred into a third separating unit 33 (the so-called depropanizer). In the third separating unit 33 a C3 fraction (reference numeral C3) is separated from the C3plus fraction and subjected to another hydrotreatment process 42, to convert the propylene contained in the C3 fraction into propene. Then the C3 fraction is separated in a C3 separating unit 36 into propene (reference numeral C3H6) and propane (reference numeral C3H8). The latter may be subjected to the steam cracking process 10 once more as recycle stream E in one or more cracking furnaces 11 to 13, separately or with other streams.

In the third separating unit 33 a liquid C4plus fraction (reference numeral C4+) remains, which is transferred into a fourth separating unit 34 (the so-called Debutanizer). In the fourth separating unit 34 a C4 fraction (reference numeral C4, referred to here as the C4 hydrocarbon stream) is separated from the C4plus fraction. A liquid C5plus fraction remains (reference numeral C5+).

It will be understood that all the fractions described can also be subjected to suitable after-treatment steps. For example, 1,3-butadiene may be separated from the C4 hydrocarbon stream, as described below. Also, additional recycle streams may be used which may be subjected to the steam cracking process 10 analogously to the recycle streams D and E.

FIG. 1B shows the course of an alternative method of producing hydrocarbons by steam cracking according to the prior art in the form of a schematic flow diagram. Once again, the core of the method is a steam cracking process 10 which may be carried out using one or more cracking furnaces 11 to 13. In contrast to the method shown in

FIG. 1A the cracking gas C here is subjected to an alternative separation process 30 according to the principle of “Deethanizer First”.

In the separation process 30 a C2minus fraction (reference numeral C2-), which may predominantly contain methane, ethane, ethylene and acetylene and, if it has not already been eliminated, hydrogen as well is first separated in gaseous form from the cracking gas C in a first separating unit 37. The C2minus fraction as a whole is subjected to a hydrotreatment process 43, to convert the acetylene it contains into ethylene. Then a C1 fraction is separated from the C2minus fraction in a C2minus separating unit 38 and further used as described above. A C2 fraction remains which is separated in a C2 separating unit 35 as above into ethylene and ethane. The latter may be subjected again to the steam cracking process 10 as a recycle stream D in one or more cracking furnaces 11 to 13. In the first separating unit 37 a liquid C3plus fraction remains which is treated in the separating units 33 to 36 and the hydrotreatment unit 42, as explained with reference to FIG. 1.

The skilled man will be familiar with numerous other process variants, for example from the article “Ethylene” in Ullmann's Encyclopedia of Industrial Chemistry mentioned hereinbefore, which differ in the preparation of the cracking gas C and/or the separation process used.

The C4 hydrocarbon stream may also be subjected to the steam cracking process 10 again in parts as a corresponding recycle stream in one or more of the cracking furnaces n to 13. Particularly when mild cracking conditions are used, however, branched C4 compounds (iso-C4 compounds) contained in the C4 hydrocarbon stream may be converted to a lesser extent than n-C4 compounds and are therefore once again found to a large extent in the cracking gas stream C. The iso-C4 compounds are therefore circulated many times through a corresponding apparatus. The consequence of such a mild steam cracking process is thus a significant increase in the amount of some product fractions, in this case the iso-C4 compounds, and a consequent reduction in the concentration of high value products present, such as 1,3-butadiene in this case, as a result of corresponding dilution effects. This makes the high value products more difficult and expensive to recover. In other words, the iso-C4 compounds contribute practically nothing to the formation of 1,3-butadiene, by virtue of their structure. The formation of a relative large quantity of largely worthless methane is unavoidable, particularly when the iso-C4 compounds are recycled until completely converted.

Thus, if C4 hydrocarbon streams are cracked with iso-C4 compounds, regardless of their origin, under mild or very mild conditions, this again results in C4 product fractions of relatively large amounts with at the same time a low 1,3-butadiene concentration.

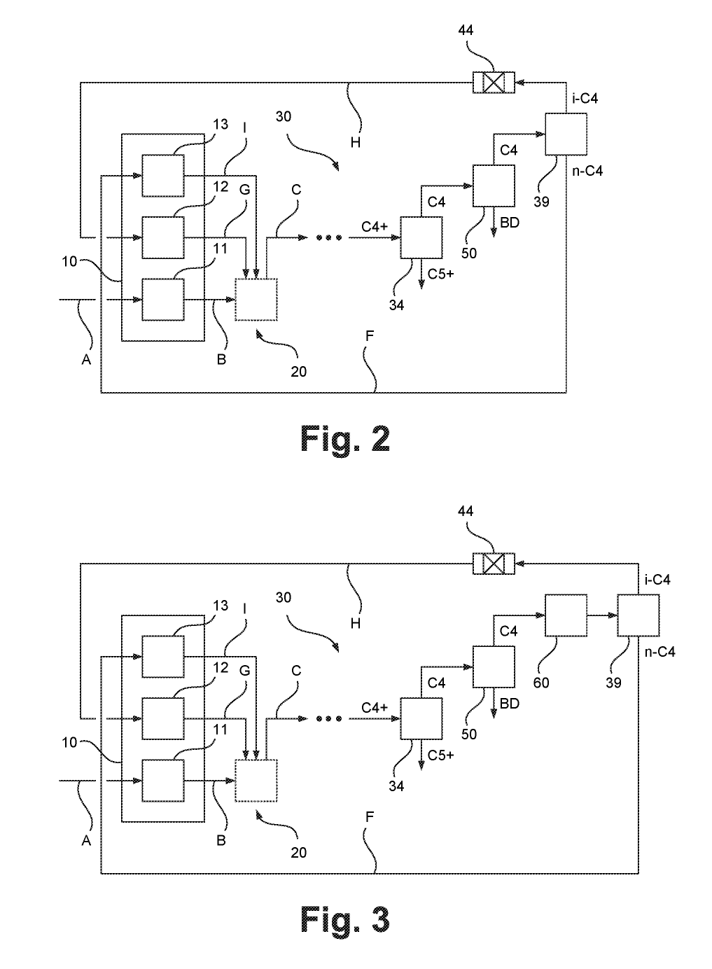

FIG. 2 shows the course of a method for producing hydrocarbons by steam cracking according to one embodiment of the invention in the form of a schematic flow diagram.

Here again, the core of the method is a steam cracking process 10 which may be carried out using cracking furnaces 11 to 13. To illustrate the universal usability of the method shown here, the recovery of a C4plus fraction from the cracking gas C is not shown; however, this may be carried out as shown in FIGS. 1A or 1B or in any other manner known in the art. In the example shown here, the C4plus fraction is supplied to a separating unit 34 which operates as described above. However, if no or only a few C5plus hydrocarbons are formed in a steam cracking process, the use of this separating unit 34 could also be dispensed with. A C4 hydrocarbon stream may, however, also be provided from outside the apparatus, e.g. from a refinery.

A C4 hydrocarbon stream obtained for example from the separating unit 34 may be fed into a 1,3-butadiene recovery unit 50 in which 1,3-butadiene, referred to here as BD, is extracted. Here, 1,3-butadiene represents one of the desired high-value products, the remaining components of the C4 hydrocarbon stream C4 are predominantly of lower economic value and “dilute” the desired 1,3-butadiene, making it more difficult to extract.

According to the embodiment shown, the invention envisages separating iso-C4 and n-C4 compounds (reference numerals i-C4 and n-C4), i.e. branched and unbranched C4 compounds, from one another in e separating unit 39 and recovering corresponding partial streams. The partial stream that predominantly contains the iso-C4 compounds is referred to here as the iso-C4 partial stream. This may be recycled as recycle stream H and either subjected once again to the steam cracking process 10 or to another steam cracking process implemented separately from the steam cracking process 10. Preferably, the iso-C4 partial stream is subjected to severe cracking conditions, for which the cracking furnace 12 is designed in this case. Hydrogenation of iso-butene may be carried out beforehand, as illustrated by block 44. A stream G removed from the cracking furnace 12 may be added, for example, to the cracking gas C, optionally after it has also previously been subjected to the preparation process 20.

The partial stream which predominantly contains the n-C4 compounds and is referred to here as the n-C4 partial stream may be recycled as recycle stream F and once again subjected either to the steam cracking process 10 or here again to another steam cracking process implemented separately from the steam cracking process 10. Preferably, the n-C4 compounds are subjected to mild to very mild cracking conditions, for which the cracking furnace 13 is designed in this case. A stream I removed from the cracking furnace 13 may be added, for example, to the cracking gas C, optionally after the latter has also previously been subjected to the preparation process 20.

FIG. 3 shows the course of a process for producing hydrocarbons by steam cracking according to another embodiment of the invention in the form of a schematic flow diagram. Once again, the core of the process is a steam cracking process 10 which can be carried out using cracking furnaces 11 to 13. Here, too, to illustrate the universal usability of the method shown here, the recovery of a C4plus fraction from the cracking gas C is not shown; however, this may be carried out as shown in FIGS. 1A or 1B or in any other manner known in the art. In the example shown here as well, the C4plus fraction is supplied to a separating unit 34 which operates as described above. Again, if no or only a few C5plus hydrocarbons are formed in a steam cracking process, the use of this separating unit 34 could also be dispensed with. A C4 hydrocarbon stream may, however, also be provided from outside the apparatus, e.g. from a refinery.

A C4 hydrocarbon stream obtained for example from the separating unit 34 may be fed into a 1,3-butadiene recovery unit 50 in which 1,3-butadiene, referred to here as BD, is extracted. Here, 1,3-butadiene represents one of the desired high-value products; the remaining components of the C4 hydrocarbon stream C4 are predominantly of lower economic value and “dilute” the desired 1,3-butadiene, making it more difficult to extract.

According to the embodiment shown, the invention provides that the C4 hydrocarbon stream is supplied to a hydroisomerisation reactor 60 downstream of the butadiene recovery unit 50, which is once again designated C4, and i-butene is at least partially converted therein to form 2-butene.

Here again, it is envisaged that iso-C4- and n-C4 compounds be separated from one another in a separating unit 39 and corresponding partial streams (n-C4 partial stream and iso-C4 partial stream) be obtained. The invention may also encompass only the recovery of the n-C4 partial stream, while the iso-C4 partial stream or compounds contained therein may be piped out of the apparatus.

The iso-C4 partial stream may be recycled as recycle stream H and either re-subjected to the steam cracking process 10 or subjected to another steam cracking process implemented separately from the latter, as explained above. Hydrogenation of iso-butene may also be carried out, as illustrated by block 44. The n-C4 partial stream may also be recycled as recycle stream F and either re-subjected to the steam cracking process 10 or subjected to another steam cracking process implemented separately from the latter, as explained above.

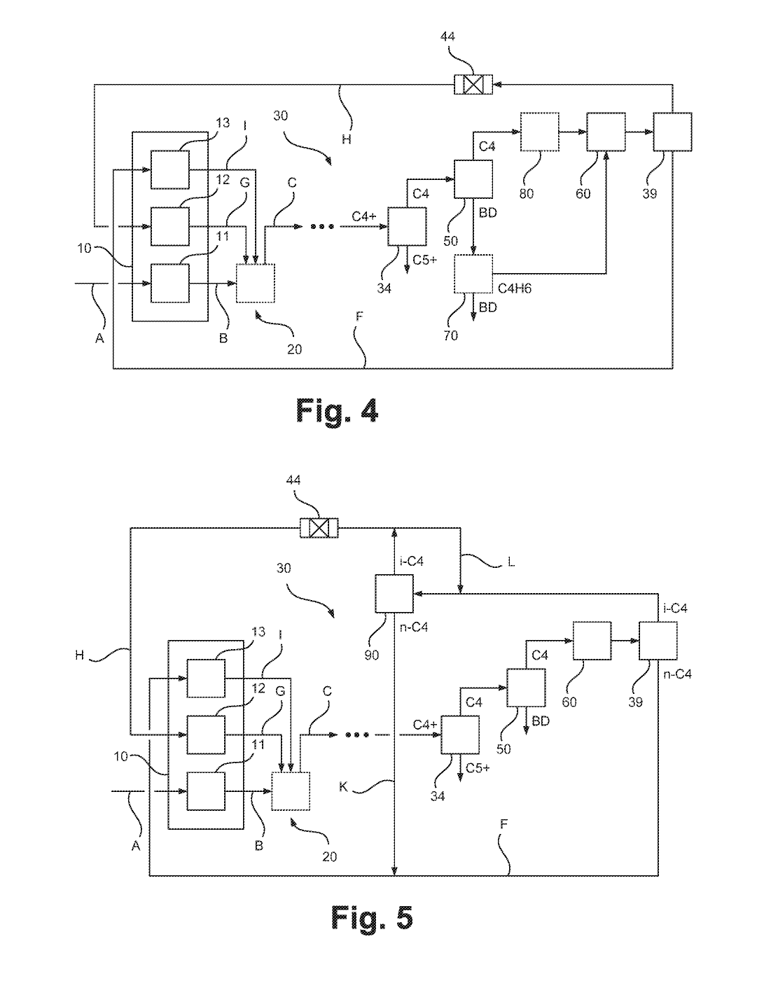

FIG. 4 shows the course of a method for producing hydrocarbons by steam cracking according to another embodiment of the invention in the form of a schematic flow diagram.

Compared with FIG. 3, an additional unit 70 is explicitly shown, which is designed to separate off unwanted coextracted components such as C4-acetylenes (reference C4H6) during the extraction of butadiene in the butadiene recovery unit 50, for example, and feed them into the hydroisomerisation reactor 60 as well. In the practical embodiment, this unit 70 is integrated in the butadiene recovery unit 50, in particular, and can also be provided as a part of the butadiene recovery unit 50 shown in FIG. 3. Alternatively or additionally, a device 80 may be provided which reacts iso-butene in the C4 hydrocarbon stream at least partially to form methyl-tert-butylether after the separation of the 1,3-butadiene and also separates the methyl-tert-butylether from the hydrocarbon stream (not shown). Alternatively it is also possible to form ethyl-tert-butylether.

FIG. 5 shows the course of a method for producing hydrocarbons by steam cracking according to another embodiment of the invention in the form of a schematic flow diagram. Once again, the core of the method is a steam cracking process 10 which can be carried out using cracking furnaces 11 to 13. For further details of the progress of the method and the devices used, reference may be made to the explanations of FIGS. 2 to 4.

The iso-C4 partial stream from the separating unit 39 is supplied here to a skeleton isomerisation reactor 90, which is designed to react at least some of the iso-C4 compounds contained in the iso-C4 partial stream to form the corresponding n-C4 compounds. At this stage, or in a subsequent separating step, a partial stream with predominantly (unconverted) iso-C4 compounds (reference numeral i-C4) and a partial stream with predominantly n-C4 compounds are once again obtained. The latter may, for example, be combined as stream K with stream F which contains the n-C4 compounds contained in the separating unit 39, and subjected to the steam cracking process 10, the cracking furnace 13 operating under mild cracking conditions, for example. The iso-C4 compounds which are not reacted in the skeleton isomerisation may also be partly or completely recycled into the skeleton isomerisation reactor 90, as illustrated with stream L, to achieve successive substantial or total conversion. However, it is also possible for a stream H formed therefrom to be cracked, optionally after hydrogenation according to block 44 (for example under severe cracking conditions in the cracking furnace 12).

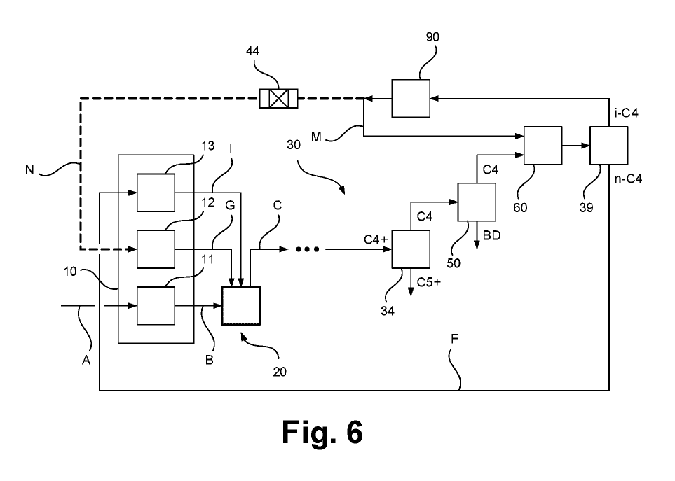

FIG. 6 shows the course of a method for producing hydrocarbons by steam cracking according to another embodiment of the invention in the form of a schematic flow diagram.

In contrast to FIG. 5 a stream which has not yet been separated into n- and iso-C4 compounds is removed from the skeleton isomerisation reactor 90. At least part of this may be recycled as stream M via the hydroisomerisation reactor 60 into the separating unit 39, the eventual result being that the unconverted iso-C4 compounds in the skeleton isomerisation reactor 90 are recycled to achieve substantial or total conversion. If necessary, however, a stream N represented by dashed lines may also be cracked, optionally after hydrogenation according to block 44 (for example under severe cracking conditions in the cracking furnace 12).

Although not shown here, it will be understood that additional recycle streams or fresh feeds may be supplied to the cracking furnaces 11 to 13.

The Figures described above show partial aspects which may each be used in different combinations with one another.

1. Method for producing hydrocarbon products, comprising:
a) providing a C4 hydrocarbon stream (C4) which comprises at least 80% branched and unbranched hydrocarbons each having four carbon atoms, and
b) recovering an n-C4 partial stream (n-C4) which comprises at least 80% unbranched hydrocarbons with four carbon atoms, and an iso-C4 partial stream (i-C4) which predominantly comprises branched hydrocarbons with four carbon atoms, from the C4 hydrocarbon stream (C4) or a stream derived therefrom,
characterised by
c) steam cracking at least part of the n-C4 partial stream (n-C4) or a stream derived therefrom at a cracking severity at which at least 50% and not more than 92% of n-butane contained therein is converted.

2. Method according to claim 1, characterised by:
reacting at least some of the branched hydrocarbons contained in the iso-C4 partial stream (i-C4) to form unbranched hydrocarbons by skeleton isomerisation.

3. Method according to claim 1 or 2, characterised by:
reacting at least some of the i-butene contained in the C4 hydrocarbon stream (C4) to form 2-butene by hydroisomerisation before recovery of the n-C4 and iso-C4 partial stream (n-C4, i-C4) according to b).

4. Method according to one of the preceding claims, characterised by:
steam cracking at least some of the iso-C4 partial stream (iso-C4) or a stream derived therefrom at a cracking severity at which more than 91% of the iso-butane contained therein is converted.

5. Method according to one of the preceding claims, wherein during the steam cracking according to c) a cracking severity is used at which less than 90%, 88%, 86%, 84%, 82%, 80%, 78%, 76%, 74%, 72%, 70% or 65% and more than 50% or 60% of the n-butane is converted.

6. Method according to one of the preceding claims, wherein the iso-C4 partial stream or a stream derived therefrom (i-C4) is at least partially subjected to a hydrogenation process.

7. Method according to one of the preceding claims, wherein the C4 hydrocarbon stream (C4) provided according to a) is at least partially produced from at least one cracking gas (C) obtained by steam cracking according to c).

8. Method according to one of the preceding claims, wherein the C4 hydrocarbon stream (C4) provided according to a) is at least partially produced from a cracking gas (C) which is formed by steam cracking a fresh feed (A), particularly from one or more petroleum fractions, petroleum gas components with two to four carbon atoms and/or petroleum gas condensates.

9. Method according to one of the preceding claims, wherein the C4 hydrocarbon stream (C4) provided according to a) is at least partially formed from a fresh feed (A) which has not been subjected to a steam cracking process beforehand, particularly from one or more petroleum fractions, petroleum gas components with two to four carbon atoms and/or petroleum gas condensates and/or at least one product of a refinery process.

10. Method according to one of the preceding claims, wherein the steam cracking is carried out using a quantity of steam of 0.4 kg/kg, particularly 0.2 to 0.7 kg/kg.

11. Method according to one of the preceding claims, wherein the steam cracking is carried out in at least one cracking furnace (11, 12, 13), which is supplied with at least one other furnace feed (A) in the form of at least one recycle stream and/or at least one fresh feed.

12. Method according to one of the preceding claims, wherein 1,3-butadiene (BD) is separated from the C4 hydrocarbon stream (C4) before the recovery of the n-C4 and iso-C4 partial streams (i-C4, n-C4) according to b).

13. Method according to claim 12, wherein, after the separation of the 1,3-butadiene (BD), isobutene contained in the hydrocarbon stream (C4) is at least partially reacted to form a tert-butylether and the tert-butylether is also separated from the hydrocarbon stream (C4).

14. Method according to one of the preceding claims, wherein at least one other stream, particularly a stream containing butyne and/or hydrocarbons with five carbon atoms, is fed into the C4 hydrocarbon stream (C4).
